(19) United States
(12) Patent Application Publication
(10) Pub. No.: US 2011/0274751 A1
(43) Pub. Date: Nov. 10, 2011

(54) TRIMETAZIDINE FORMULATION WITH DIFFERENT RELEASE PROFILES

(75) Inventors: Umit Cifter, Istanbul (TR); Ali Turkyilmaz, Istanbul (TR); Nur Pehlivan Akalin, Istanbul (TR); Aylin Yildirim, Istanbul (TR); Levent Oner, Ankara (TR)

(73) Assignee: Sanovel Ilac Sanayi Ve Ticaret Anonim Sirketi, Istanbul (TR)

(21) Appl. No.: 13/099,878

(22) Filed: May 3, 2011

(30) Foreign Application Priority Data
May 4, 2010 (TR) 201003511

(51) Int. Cl. A61K 9/24; A61P 9/10; A61P 9/00; A61K 9/00; A61K 31/495

(52) U.S. Cl. 424/465; 424/400; 514/252.12; 424/472

(57) ABSTRACT

A multilayered solid oral pharmaceutical formulation of trimetazidine or a pharmaceutically acceptable salt or polymorph of trimetazidine wherein one layer of said formulation provides controlled release, while the other layer provides immediate release.

### CROSS REFERENCE TO RELATED APPLICATIONS

This application is based upon Turkish Patent Application No. TR201003511, filed May 5, 2010, under relevant sections of 35 USC §119, the entire contents of this application being incorporated by reference herein.

### FIELD OF THE INVENTION

The present invention relates to formulations of trimetazidine or a pharmaceutically acceptable salt of trimetazidine. The present invention more particularly relates to a formulation of trimetazidine, providing immediate release and controlled release concomitantly.

### BACKGROUND OF THE INVENTION

Trimetazidine, with the chemical name 1-(2,3,4-trimethoxybenzyl)piperazine, has the following chemical structure of Formula I.

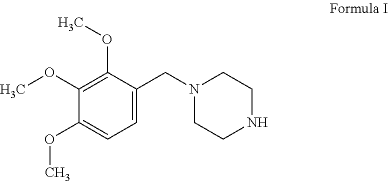

Trimetazidine is an anti-ischemic agent and used in the prophylaxis and treatment of angina pectoris and vertigo. It has considerably high solubility and is rapidly absorbed upon administration. Its half-life is 6 hours.

There are various patent applications available in relation to trimetazidine. For example, EP0533579 discloses trimetazidine derivatives and a process for obtaining the same.

EP0673649 discloses a pharmaceutical composition providing prolonged release of trimetazidine or a pharmaceutically acceptable salt thereof. Prolonged release of trimetazidine is controlled by means of a reservoir system in this composition. In this reservoir system, a mixture of a water-insoluble polymer and a plasticizer is in the form of a film that coats tablets or minigranules containing trimetazidine or an addition salt thereof, together with a pharmaceutically acceptable acid. The plasticizing agent here is not hydrogenated oil.

EP1108424 discloses a matrix object for prolonged release of trimetazidine or a pharmaceutically acceptable salt thereof, of which the prolonged release is controlled by using a polymer derived from cellulose, selected among hydroxypropyl methyl cellulose, hydroxyethyl cellulose, hydroxymethyl cellulose, methyl cellulose, hydroxypropyl cellulose.

EP1448173 discloses a process for preparing a “once a day” drug containing 60 mg trimetazidine dihydrochloride and having an pH-independent in vitro release, as well as a novel composition prepared by this process. This composition is composed of uniformly-shaped and sized microbeads.

Various formulations have been developed with trimetazidine. Reviewed generally, it can be concluded that there are various conventional tablets and controlled-release formulations of trimetazidine molecule. The posology of such formulations is in the form of administering 3 conventional products or 2 controlled-release products per day. “Once a day” administration of doses gains high importance and need with respect to patients. EP 1 448 173 discloses a controlled-release tablet containing 60 mg trimetazidine dihydrochloride and claims to treat patients, needing such treatment, by administering one dose thereof daily. A certain period of time is required, however, to the onset of the effect of said formulation. This is not desirable for the patient. A quick onset of the treatment process directly affects patient life quality.

Trimetazidine dihydrochloride is highly-soluble in water. It is therefore quite difficult to develop a formulation of highly-soluble trimetazidine dihydrochloride with release profiles of desired quality. For instance, such release profiles may show fluctuations. On the other hand, polymers providing proper release profiles impose problems, such as sticking to punches and other equipment during production, due to their viscosities.

Considering the aforesaid problems, it is obvious that a novelty is needed in both providing production convenience in the art of “once a day” dose formulations containing trimetazidine dihydrochloride, and ensuring convenient release profiles for the same.

### SUMMARY OF THE INVENTION

The present invention provides a trimetazidine formulation, eliminating all aforesaid problems and brining additional advantages to the relevant prior art.

Accordingly, the main object of the present invention is to provide a trimetazidine formulation administered orally once or twice per day.

Another object of the present invention is to reduce the treatment onset time, while said formulation provides a 24-hour effect.

A further object of the present invention is to prevent the production items from sticking to contact surfaces of machines and equipment.

A solid oral pharmaceutical formulation comprising trimetazidine or a pharmaceutically acceptable salt or polymorph of trimetazidine and having multiple layers has been developed to carry out all objects, referred to above and emerging from the following detailed description.

According to a preferred embodiment of the present invention, this novelty is characterized in that one layer of said formulation provides controlled release, whereas the other layer provides immediate release.

According to a preferred embodiment of the present invention, this formulation comprises one or more excipients.

According to an embodiment of the present invention, this formulation comprises at least one intermediate layer.

According to a preferred embodiment of the present invention, the trimetazidine is trimetazidine dihydrochloride.

According to a preferred embodiment of the present invention, the amount of trimetazidine dihydrochloride in the immediate-release layer is not more than 25% and is preferably 10% by weight of the total amount of trimetazidine dihydrochloride present in the tablet.

According to a preferred embodiment of the present invention, the controlled-release providing agent contains one or a mixture of polymethacrylate, glyceryl behenate, polyvinylpyrrolidone (povidone), cross-linked polyvinylpyrrolidone, hydroxypropyl methyl cellulose (HPMC), hydroxypropyl cellulose (HPC), carboxymethyl cellulose (CMC), methyl cellulose (MC), ethyl cellulose (EC) and other cellulose derivatives, polyethylene oxide and gelatin; but this controlled-release agent is preferably polyethylene oxide. The controlled-release agent is used in the outer granule phase.

According to a preferred embodiment of the present invention, the ratio of trimetazidine dihydrochloride in said controlled-release layer to polyethylene oxide is between 0.05 to 10, preferably 0.1 to 5, and more preferably 0.2 to 0.8.

According to a preferred embodiment of the present invention, the excipients comprise at least one or a mixture of binders, diluents, disintegrants, glidants, and lubricants.

According to a preferred embodiment of the present invention, the binder comprises at least one or a mixture of cellulose derivatives, such as polyvinylpyrrolidone (povidone), hydroxypropyl methyl cellulose, hydroxypropyl cellulose, carboxymethyl cellulose, methyl cellulose, ethyl cellulose; and gelatin; but is preferably polyvinylpyrrolidone.

According to a preferred embodiment of the present invention, the diluents comprise at least one or a mixture of lactose, starch, mannitol, calcium hydrogen phosphate dihydrate, dicalcium hydrogen phosphate anhydrate, calcium phosphate trihydrate, silicium dioxide and glucose; but is preferably selected from calcium hydrogen phosphate dihydrate and dicalcium hydrogen phosphate anhydrate.

According to a preferred embodiment of the present invention, the glidants comprise at least one or a mixture of colloidal silicone dioxide, talc, aluminum silicate, and magnesium silicate; but is preferably colloidal silicone dioxide.

According to a preferred embodiment of the present invention, the formulation comprises magnesium stearate as a lubricant.

Another preferred embodiment according to the present invention consists of:

a. an immediate release layer having:

a) trimetazidine dihydrochloride at 0.1 to 10% by weight;

b) dicalcium hydrogen phosphate anhydrate at 5 to 90% by weight;

c) colloidal silicone dioxide at 0.1 to 5% by weight;

d) magnesium stearate at 0.1 to 5% by weight; and

e) red iron oxide at 0.01 to 5% by weight; and

b. a controlled release layer having:

a) trimetazidine dihydrochloride at 2.5 to 50% by weight;

b) polyethylene oxide at 2 to 90% by weight;

c) polyvinylpyrrolidone at 0.1 to 20% by weight;

d) calcium hydrogen phosphate dihydrate at 5 to 90% by weight;

e) colloidal silicone dioxide at 0.1 to 5% by weight; and

f) magnesium stearate at 0.1 to 5% by weight.

A further preferred embodiment according to the present invention provides a method for preparing a pharmaceutical formulation, this method comprising the steps of:
a) sieving and then mixing trimetazidine dihydrochloride, polyvinylpyrrolidone and calcium hydrogen phosphate dihydrate in a high-shear mixer;
b) granulating the mixture from above with sufficient amount of water;
c) sieving the wet granules formed, then drying the same and sieving back the dried granules;
d) adding polyethylene oxide and colloidal silicone dioxide into the granules obtained and mixing the latter;
e) sieving magnesium stearate and mixing it into the resultant mixture to obtain the controlled-release phase;
f) sieving and mixing together trimetazidine dihydrochloride, dicalcium hydrogen phosphate anhydrate, colloidal silicone dioxide and red iron oxide;
g) sieving magnesium stearate and mixing it into the resultant mixture to obtain the immediate-release phase; and
h) compacting the controlled-release phase and immediate-release phase to provide a bilayer tablet.

### DETAILED DESCRIPTION OF THE INVENTION

### Example 1

Trimetazidine MR 35 mg Tablet Unit Formula (mg)

| | Immediate-release | Controlled-release |
|---|---|---|
| | phase 10% | phase (90%) |
| | | Inner Phase |
| Trimetazidine dihydrochloride | 3.5 | 31.5 |
| PVP K-90 F | — | 3.0 |
| Calcium hydrogen phosphate | — | 50.0 |
| dihydrate | | |
| | | Outer Phase |
| Polyethylene oxide WSR 303 | — | 90.0 |
| Dicalcium hydrogen phosphate | 37.5 | — |
| anhydrate | | |
| Magnesium stearate | 0.4 | 1.8 |
| Colloidal silicone dioxide | 0.2 | 0.9 |
| Red iron oxide | 0.2 | — |

### Producing the Controlled-Release Phase

Trimetazidine dihydrochloride, polyvinylpyrrolidone and calcium hydrogen phosphate dihydrate are sieved and mixed in a high-shear mixer. The resulting mixture is then granulated with an adequate amount of water. Wet granules formed are sieved, then dried and the dried granules are sieved back. Polyethylene oxide and colloidal silicone dioxide are added into and mixed together with the granules obtained. Polyethylene oxide providing controlled release is used in the outer granule phase. When polyethylene oxide is included into wet granules, it leads to problems of sticking over the machinery and equipment surfaces. Thus, polyethylene oxide is used in the outer phase to prevent this problem so that a substantial potential problem is avoided. Magnesium stearate is added into and mixed with this mixture to produce the controlled-release phase.

### Producing the Immediate-Release Phase

Trimetazidine dihydrochloride, dicalcium hydrogen phosphate anhydrate, colloidal silicone dioxide and red iron oxide are mixed in a separate kettle. To this obtained mixture, sieved magnesium stearate is added to directly give the immediate-release phase. The controlled-release phase and the immediate-release phase are compacted to provide a bilayer tablet.

The following table provides comparative data of dissolution profiles of Vastarel MR and the formulation coded 01C10 developed by the inventors.

| | Vastarel | 01C10 |
|---|---|---|
| Time (minute) | 0.1N HCl | 0.1N HCl |
| 0 | 0 | 0 |
| 15 | 16 | 19 |
| 30 | 24 | 29 |
| 45 | 30 | 37 |
| 60 | 36 | 44 |
| 120 | 53 | 65 |
| 180 | 67 | 80 |
| 240 | 79 | 88 |
| 300 | 87 | 92 |
| 360 | 93 | 94 |
| 420 | 99 | 99 |
| 480 | 100 | 99 |

### Example 2

Trimetazidine MR 35 mg Tablet Unit Formula (mg)

| | Immediate-release | Controlled-release |
|---|---|---|
| | phase (10%) | phase (90%) |
| | | Inner Phase |
| Trimetazidine dihydrochloride | 3.5 | 31.5 |
| PVP K-90 F | — | 3.0 |
| Calcium hydrogen phosphate | — | 70.0 |
| dihydrate | | |
| | | Outer Phase |
| Glyceryl behenate | — | 70.0 |
| Dicalcium hydrogen phosphate | 37.5 | — |
| anhydrate | | |
| Magnesium stearate | 0.4 | 1.8 |
| Colloidal silicone dioxide | 0.2 | 0.9 |
| Red iron oxide | 0.2 | — |

Generally, when some controlled release agents are included into wet granules, it leads to problems of sticking over the machinery and equipment surfaces. Controlled release agents are used in the outer phase so that these agents prevent problems of sticking over the machinery and equipment. According to a preferred embodiment of the present invention, the controlled-release providing agent comprises at least one or a mixture of polymethacrylate, glyceryl behenate, polyvinylpyrrolidone (povidone), cross-linked polyvinylpyrrolidone, hydroxypropyl methyl cellulose (HPMC), hydroxypropyl cellulose (HPC), carboxymethyl cellulose (CMC), methyl cellulose (MC), ethyl cellulose (EC) and other cellulose derivatives, polyethylene oxide and gelatin; said controlled-release agent is preferably selected from glyceryl behenate, hydroxypropyl methyl cellulose (HPMC), hydroxypropyl cellulose (HPC), carboxymethyl cellulose (CMC), methyl cellulose (MC), ethyl cellulose (EC) and other cellulose derivatives, polyethylene oxide and gelatin. According to one version, the controlled-release agent is preferably polyethylene oxide.

These studies conducted at 0.1N HCl show that the 01C01 coded product according to the present invention dissolves more, particularly during the first 3 hours. Thus, the time for the treatment onset is shortened.

This formulation is embodied for 35 and 70 mg trimetazidine tablets. 35 mg and 70 mg tablets are developed to provide drug activity up to 24 hours.

This invention has surprisingly provided a bilayer tablet formulation containing trimetazidine dihydrochloride, which does not stick to machinery during production and shows desired release profiles. The amounts of immediate-release and controlled-release phases in said bilayer tablet are such determined that 35 and 70 mg formulations are obtained of convenient and desired quality, which provide drug release up to 24 hours. The amount of trimetazidine dihydrochloride in said immediate-release layer is not more than 25% and is preferably 10% by weight of the total amount of trimetazidine dihydrochloride present in the tablet.

The formulation developed is used in treating angina pectoris, chorioretinal vascular disorders, tinnitus, vertigo, and meniere's syndrome.

Although the tablet obtained is bilayered, it is alternatively possible to design said tablet with more layers as well. It is possible to place one or more intermediate layers between the layers that contain the active agent in order to combine the layers, such intermediate layers possibly including an active agent or not including an active agent, and providing immediate release or controlled release. Whilst both layers including the active agent may provide controlled release or immediate release, at least one thereof may provide immediate release while the others provide controlled, prolonged, and retarded release.

It is additionally possible to use the following additional auxiliaries in the formulation.

Suitable binders include, but are not restricted to, at least one or a mixture of polyvinylpyrrolidone, gelatin, sugars, glucose, natural gums, gums, synthetic celluloses, polymethacrylate, hydroxypropyl methyl cellulose, hydroxypropyl cellulose, carboxymethyl cellulose, methyl cellulose, and other cellulose derivatives.

Suitable glidants include, but are not restricted to, at least one or a mixture of colloidal silicone dioxide, talc, and aluminum silicate.

Suitable lubricants include, but are not restricted to, at least one or a mixture of sodium stearil fumarat, magnesium stearate, polyethylene glycol, stearic acid, metal stearates, boric acid, sodium chloride benzoate and acetate, sodium or magnesium lauryl sulfate.

Suitable disintegrants include, but are not restricted to, at least one or a mixture of sodium starch glycolate, croscarmellose sodium, crospovidone, sodium alginate, gums, starch, and magnesium aluminum silicate.

Suitable surface active agents include, but are not restricted to, at least one or a mixture of sodium lauryl sulfate, dioctyl sulfosuccinate, polysorbates and polyoxyethylene alkyl esters and ethers thereof, glyceryl monolaurate saponins, sorbitan laurate, sodium lauryl sulfate, and magnesium lauryl sulfate.

Suitable coating agents include, but are not restricted to hydroxypropyl methyl cellulose, polyethylene glycol, polyvinylpyrrolidone, polyvinylpyrrolidone-vinyl acetate copolymer (PVP-VA), polyvinyl alcohol and other polymers, and all kinds of Opadry™, as well as pigments, dyes, titanium dioxide and iron oxide and talc.

The protection scope of the present invention is set forth in the following claims and cannot be restricted to the illustrative disclosures given above, under the detailed description. Any alternative embodiments to be produced by persons skilled in the art according to the basic principles, which are under the protection scope as set forth in the claims, shall be an infringement of the present invention.

1. A multilayered solid oral pharmaceutical formulation of trimetazidine or a pharmaceutically acceptable salt or polymorph of trimetazidine, comprising a first layer of said formulation providing controlled release wherein a controlled-release providing agent is used in an outer granule phase, while a second layer provides immediate release.

2. The pharmaceutical formulation according to claim 1, further comprising at least one or more excipients.

3. The pharmaceutical formulation according to claim 1, comprising preferably at least one intermediate layer between said first and second layers.

4. The pharmaceutical formulation according to claim 1, said tablet containing trimetazidine dihydrochloride wherein the amount by weight of trimetazidine dihydrochloride in said immediate-release layer is not more than 25% by weight of the total amount of trimetazidine dihydrochloride present in the tablet.

5. The pharmaceutical formulation according to claim 1, said tablet containing trimetazidine dihydrochloride wherein the amount by weight of trimetazidine dihydrochloride in said immediate-release layer is not more than 10% by weight of the total amount of trimetazidine dihydrochloride present in the tablet.

6. The pharmaceutical formulation according to claim 1, including at least one controlled-release providing agent comprising at least one or a mixture of polymethacrylate, glyceryl behenate, polyvinylpyrrolidone (povidone), cross-linked polyvinylpyrrolidone, hydroxypropyl methyl cellulose (HPMC), hydroxypropyl cellulose (HPC), carboxymethyl cellulose (CMC), methyl cellulose (MC), ethyl cellulose (EC) and other cellulose derivatives, polyethylene oxide and gelatin.

7. The pharmaceutical composition according to claim 6, wherein said controlled-release agent is selected from at least one of the group consisting of glyceryl behenate, hydroxypropyl methyl cellulose (HPMC), hydroxypropyl cellulose (HPC), carboxymethyl cellulose (CMC), methyl cellulose (MC), ethyl cellulose (EC) and other cellulose derivatives, polyethylene oxide and gelatin.

8. The pharmaceutical formulation according to claim 6, wherein the ratio of trimetazidine dihydrochloride in said controlled-release layer to polyethylene oxide is between 0.05 to 10.

9. The pharmaceutical formulation according to claim 6, wherein the ratio of trimetazidine dihydrochloride in said controlled-release layer to polyethylene oxide is between 0.1 to 5.

10. The pharmaceutical formulation according to claim 6, wherein the ratio of trimetazidine dihydrochloride in said controlled-release layer to polyethylene oxide is between 0.2 to 0.8.

11. The pharmaceutical formulation according to claim 2, wherein said at least one excipient comprises at least one or a mixture of binders, diluents, disintegrants, glidants, and lubricants.

12. The pharmaceutical formulation according to claim 11, wherein said binder contains at least one or a mixture of polyvinylpyrrolidone (povidone), hydroxypropyl methyl cellulose, hydroxypropyl cellulose, carboxymethyl cellulose, methyl cellulose, ethyl cellulose and other cellulose derivatives and gelatin.

13. The pharmaceutical formulation according to claim 12, wherein said binder is polyvinylpyrrolidone.

14. The pharmaceutical formulation according to claim 11, wherein said diluent contains at least one or a mixture of lactose, starch, mannitol, calcium hydrogen phosphate dihydrate, dicalcium hydrogen phosphate anhydrate, calcium phosphate trihydrate, silicium dioxide and glucose.

15. The pharmaceutical formulation according to claim 11, wherein said diluent is selected from at least one of calcium hydrogen phosphate dihydrate and dicalcium hydrogen phosphate anhydrate.

16. The pharmaceutical formulation according to claim 11, wherein said glidant comprises at least one or a mixture of colloidal silicone dioxide, talc, aluminum silicate, and magnesium silicate.

17. The pharmaceutical formulation according to claim 11, wherein the lubricant used comprises magnesium stearate.

18. A pharmaceutical formulation, consisting of:
a. an immediate-release layer having:
a) trimetazidine dihydrochloride at 0.1 to 10% by weight;
b) dicalcium hydrogen phosphate anhydrate at 5 to 90% by weight;
c) colloidal silicone dioxide at 0.1 to 5% by weight;
d) magnesium stearate at 0.1 to 5% by weight; and
e) red iron oxide at 0.01 to 5% by weight; and
b. a controlled-release layer having:
a) trimetazidine dihydrochloride at 2.5 to 50% by weight;
b) polyethylene oxide at 2 to 90% by weight;
c) polyvinylpyrrolidone at 0.1 to 20% by weight;
d) calcium hydrogen phosphate dihydrate at 5 to 90% by weight;
e) colloidal silicone dioxide at 0.1 to 5% by weight; and
f) magnesium stearate at 0.1 to 5% by weight.

19. A method for preparing a pharmaceutical formulation according to claim 1, said method comprising the steps of:
a) sieving and then mixing trimetazidine dihydrochloride, polyvinylpyrrolidone and calcium hydrogen phosphate dihydrate in a high-shear mixer;
b) granulating the mixture from above with sufficient amount of water;
c) sieving the wet granules formed, then drying the same and sieving back the dried granules;
d) adding polyethylene oxide and colloidal silicone dioxide into the granules obtained and mixing the latter;
e) sieving magnesium stearate and mixing it into the resultant mixture to obtain the controlled-release phase;
f) sieving and mixing together trimetazidine dihydrochloride, dicalcium hydrogen phosphate anhydrate, colloidal dioxide and red iron oxide;
g) sieving magnesium stearate and mixing it into the resultant mixture to obtain the immediate-release phase; and
h) compacting the controlled-release phase and immediate-release phase to provide a bilayer tablet.

20. The pharmaceutical formulation according to claim 1 for preventing or treating angina pectoris, chorioretinal vascular disorders, tinnitus, vertigo, and meniere's syndrome in mammalians and particularly in humans.
